(19) United States
(12) Patent Application Publication
(10) Pub. No.: US 2010/0098668 A1
(43) Pub. Date: Apr. 22, 2010

(54) Oncolytic Adenoviruses and Uses Thereof

(75) Inventor: Prem Seth, Glenview, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: NORTHSHORE UNIVERSITY HEALTH SYSTEM, EVANSTON (IL)

(21) Appl. No.: 12/443,642

(22) PCT Filed: Oct. 1, 2007
(86) PCT No.: PCT/US07/80098

§ 371 Date: Dec. 14, 2009

Related U.S. Application Data
(60) Provisional application No. 60/827,654, filed on Sep. 29, 2006.

(51) Int. Cl. A61K 35/76; C12N 7/00; A61P 35/00

(52) U.S. Cl. 424/93.6; 435/235.1

(57) ABSTRACT

The disclosed subject matter provides methods and materials relating to viral vectors, such as adenoviral vectors, that effectively target cancer cells and that express a protein that specifically binds to Transforming Growth Factor-β.

### GOVERNMENT INTERESTS

This work was supported by a Research Career Development Award by Evanston Northwestern Healthcare. The invention was made with U.S. government support under Department of Defense Breast Cancer Research Program grants DAMAD17-03-0703, W811XWH-06-1-0632, and W81XWH-07-1-0586, as well as NIH grants R21 CA1 1288-01 and RO1 CA 127380.

### FIELD

The subject matter of the disclosure relates generally to the treatment of cancer in its various forms. More specifically, the disclosed subject matter relates to virus-mediated cancer treatment.

### BACKGROUND

Cancer, a collection of diseases characterized by cell proliferation unchecked by cell cycle regulatory mechanisms operative in healthy cells, is widely recognized as one of the main health threats today, with 553,440 cancer-related deaths per year in the U.S. alone. Diagnoses of breast cancer, a single type of the deadly disease, average nearly 200,000 women each year, again in the U.S. alone, causing considerable mortality (American Cancer Society, 2005 Facts and Figures). The deadly nature of the disease, and the quality-of-life and economic consequences of its occurrence, have led to considerable effort by the medical community to prevent and treat the various forms of cancer. Conventional therapeutic modalities, such as surgery, radiotherapy and chemotherapy, have been joined by methodologies recently developed to exploit advances in molecular biology and, more specifically, molecular oncology. For example, progress in molecular genetics has led to the availability of nucleic acid coding regions providing an approach to the delivery of a variety of therapeutic agents, including cytotoxic compositions such as tumor suppressors and enzymes capable of converting relatively inert prodrugs to cytotoxins. Other nucleic acids encode modified polypeptides such as anti-oncogenes encoded by mutated coding regions, coding regions expressing antisense RNAs, ribozymes or siRNAs, coding regions for immunomodulators such as cytokines or tumor antigens, the latter also useful in a preventive role via vaccination. In addition, nucleic acids are available that encode anti-angiogenic products such as endostatin and angiostatin, useful in thwarting development of the blood supply to sustain tumor growth.

Effective delivery of these nucleic acids requires a suitable delivery vehicle. One prominent type of delivery vehicle is a nucleic acid vector. A type of vector that has received some attention is the viral vector, which typically provides a natural packaging system to facilitate the preparation of functional vectors and which also typically is found to contain non-essential genetic regions that can be replaced with therapeutic coding regions. Further, viruses offer the potential of providing an additional cytotoxic force in that many viruses are, or can be engineered to be, cytotoxic themselves, for example through completion of a lytic life cycle. With that potential benefit, however, comes the risk of insufficient specificity in delivering that cytotoxic effect, resulting in deleterious consequences arising from viral attack on healthy cells. Thus, the most suitable viral vector for use in anti-cancer therapy would possess sufficient efficacy in terms of cytotoxicity towards cancer cells (e.g., tumor cells) while also exhibiting sufficient specificity in terms of selectively attacking cancer cells, but not healthy cells. Although a variety of eukaryotic viruses have been explored to assess their potentials for use in delivering therapeutics, the perfect candidate has yet to emerge.

In the last several years, there has been a significant interest in using adenoviral vectors for high-level gene expression in mammalian cells (1-4). Adenovirus is a non-enveloped DNA virus capable of inducing efficient receptor-mediated endocytosis. Genetic engineering of adenovirus has revealed that it can package up to 35 kb of foreign DNA and the virus is capable of high levels of transient gene expression in both dividing and non-dividing cells. Most expression studies using adenoviral vectors have employed replication-deficient (e.g., E1-deleted) adenoviruses expressing the gene(s) of interest (1-4). An exemplary mutant adenovirus is Ad dl01/07, which contains two mutations in the gene encoding E1A. The dl01 mutation is a deletion of the region encoding amino acids 4-25 of E1A, a region that includes the p300 binding region. The dl07 mutation deletes the coding region for amino acids 111-123 of E1A, which includes the pRb binding region. Interaction of E1A with the p300 and pRb proteins is needed for effective host cell entry into the S phase of the cell cycle.

Recently, adenoviruses that selectively replicate in tumor cells with certain genetic backgrounds (e.g., tumor cells expressing mutant p53 protein) have been developed (5-7). The selective replication of oncolytic adenoviruses in tumor cells amplifies the viral titer, resulting in cell lysis. Although replicating adenoviruses have an advantage over replication-deficient adenoviruses (which do not lyse infected cells and cannot spread from cell-to-cell in a tumor mass), in vivo efficacy of even oncolytic viruses is generally insufficient for cancer therapy. Moreover, replication-competent viruses increase the risk of undesirable infection of healthy, or at least non-targeted, cells. Therefore, there is a tremendous need to enhance the effectiveness of oncolytic viruses for cancer therapy.

Transforming Growth Factor-β (TGF-β) plays an important role in late-stage tumorigenesis by stimulating tumor invasion, promoting neoangiogenesis, inducing bone metastasis, and helping cancer cells to escape immunosurveillance (9-19). In humans, TGF-β belongs to a family of proteins that contains almost 30 members, including bone morphogenic proteins, activins, and Mullerian inhibiting substance, but TGF-β itself has three mammalian isoforms (TGF-β1, TGF-β2, and TGF-β3), each with distinct functions in vivo. After TGF-β binding to Transforming Growth Factor-β Receptor II (TGFβRII), TGF-β type I receptor is recruited to the complex. Each of these receptors is a transmembrane serine/threonine kinase. The binding and recruitment allows the constitutively active TGF β RII kinase to transphosphorylate and activate the TGF-β type I receptor kinase (also known as activin receptor-like kinase). In breast cancer cells, this activation initiates a downstream response by various pathways that include SMADs, extracellular signal-regulated kinase-1, extracellular signal-regulated kinase-2, mitogen-activated protein kinases (MAPK), phosphatidylinositol 3-kinase pathways, and the activation induces transcriptional modulation of target genes (20, 21). With respect to the SMAD (homologs of C. elegans Sma and D. melanogaster MAD, or Mothers Against Decapentaplegic) protein family, activation of the TGF-β type I receptor by type II receptor-mediated phosphorylation results in propagation of the signal intracytoplasmically to the transcription machinery, which occurs by direct phosphorylation of SMAD proteins. Eight SMAD family members have been documented. In the case of TGF-βs, however, SMAD-2 and SMAD-3 (or receptor-phosphorylated SMADs) are the positive signaling SMADs. These phosphorylated SMADs heterodimerize with a common shared partner, SMAD-4. After this they translocate to the nucleus where, by recruitment of coactivator proteins, they participate in the transcriptional activation of target genes. In contrast, SMAD-6 and SMAD-7 are negative regulators. SMAD-7 binds to the TGF-β-activated receptor, where it inhibits the phosphorylation of SMAD-2 and SMAD-3, consequently downregulating the signaling pathway. SMAD-7 expression is also upregulated by TGF-β1, allowing it to act in an autoinhibitory feedback loop that shuts off TGF-β signaling. Negative control also occurs by degradation of SMADs following their ubiquitinylation by SMURF-1.

Thus, there is a tremendous need to develop viral-based therapies to treat cancers such as breast cancer that provide a cytotoxic effect on both the cells of a primary tumor and metastatic cells, such as cells found in metastases of the bone, lung, brain, and other tissues and organs of man and other animals, such as non-human mammals. Preferably, the therapeutic virus will replicate in all cancer cells, regardless of genotype, will spread intratumorally, will be effective against remote metastases, and will be amenable to the incorporation of additional tumor-targeting capacity.

### SUMMARY

A cancer gene therapy approach combining the oncolytic effects of an adenoviral vector with selective expression of a protein that specifically binds to TGF-β, such as a soluble form of the TGF-β receptor-II, alone or fused to a stabilizing peptide such as Fc (sTGFβRIIFc) is disclosed. In preferred embodiments, an adenoviral dl01/07 mutant is used because it can replicate in all cancer cells regardless of their genetic defects. A sTGFβRII cDNA was cloned in conditionally replicating adenoviral vector rAds-TRII and in a replication-deficient adenovirus Ad-sTRII. Infection of MDA-MB-231 breast cancer cells with rAds-TRII or Ad-sTRII followed by Western blot analysis indicated the expression of diffused glycosylated forms of sTGFβRII that were also secreted into the extracellular medium. The secreted proteins were shown to bind with TGF-β and antagonize TGF-β-induced, p38 mitogen-activated protein kinase activity. However, marked differences in the replication potential of rAd-sTRII and Ad-sTRII were observed in breast tumor cells. Infection of MDA-MB-231 cells with rAd-sTRII resulted in cytotoxicity and significant increase in the adenoviral titers that were comparable with a wild-type adenovirus dl/309. However, Ad-sTRII was much less toxic to the tumor cells, and the viral titers of Ad-sTRII remained relatively unchanged. These results establish that the infection of breast tumor cells with conditionally replicating adenoviral vector rAd-sTRII produced sTGFβRII that can abrogate TGF-β signaling, while maintaining the replication potential of the virus, indicating that rAd-sTRII could be a potential anticancer agent.

An oncolytic adenovirus expressing sTGFβRIIFc (Ad.sTβRFc) was constructed by homologous recombination. Infection of MDA-MB-231 and MCF-7 human breast cancer cells with Ad.sTβRFc produced sTGFβRIIFc that was released into the medium. The conditioned medium containing sTGFβRIIFc bound TGFβ-1 and inhibited TGFβ-dependent transcription in the target cells. Infection of MDA-MB-231, MCF-7, and 76NE human breast cancer cells with Ad.sTβRFc resulted in high levels of viral replication that were comparable to that of the wild-type dl309 virus. Direct injection of Ad.sTβRFc into MDA-MB-231 human breast xenograft tumors grown in nude mice resulted in significant inhibition of tumor growth, causing tumor regressions in more than 85% of the animals. These results indicate that it is possible to construct an oncolytic virus expressing sTGFβRIIFc in which both viral replication and transgene expression remain intact, and that the recombinant adenovirus is oncolytic in a human tumor xenograft model. Additional data disclosed herein establishes that these oncolytic viruses are effective in breast tumor xenograft reduction when administered intravenously. Based on these results, it is apparent that Ad.sTβRFc is useful as an anti-tumor agent.

One aspect of the disclosure is accordingly drawn to a method for treating cancer comprising delivering a therapeutically effective amount of an adenovirus expressing an E1A protein deficient in facilitating host cell transition to the S phase of growth and further comprising a coding region for a protein that specifically binds to TGF-β. In some embodiments, the adenovirus comprises a mutated E1A coding region, such as the Ad dl01/07 adenovirus. Also in some embodiments, the coding region encodes a TGF-β receptor. An exemplary TGF-β receptor is TGF-β receptor II, e.g., a soluble TGF-β receptor II. This aspect of the disclosure extends to methods wherein the protein that specifically binds to TGF-β is a fusion protein, such as a fusion between a TGF-β receptor and an F_{C} fragment of an immunoglobulin. The F_{C} fragment may be an IgG F_{C} fragment, such as an IgG1 F_{C} fragment. In some embodiments, the adenovirus is administered directly to the tumor; in other embodiments, the adenovirus is administered indirectly, e.g., by intravenous injection.

Another aspect of the disclosure is drawn to a method for treating cancer comprising delivering a therapeutically effective amount of an adenovirus comprising an essential adenoviral gene under the expression control of a tumor-specific promoter and further comprising a coding region for a protein that specifically binds to TGF-β. In some embodiments, the tumor-specific promoter is selected from the group consisting of an hTERT promoter, a modified hTERT promoter and a promoter for a small mucin-like protein. Also in some embodiments, the protein that specifically binds to TGF-β is selected from the group consisting of TGF-β receptor I, TGF-β receptor II and soluble TGF-β receptor II. Again, it is contemplated that any administration route will be useful in delivering the adenovirus, including direct administration and indirect administration, e.g., intravenous administration, and these routes are also contemplated for the aspect of the disclosure addressed immediately below.

Another aspect of the disclosure is a method for treating cancer comprising delivering a therapeutically effective amount of an adenovirus comprising a coding region for a fusion protein comprising an adenoviral fiber protein and a binding pair member that specifically interacts with a binding partner associated with a cancer cell, and further comprising a coding region for a protein that specifically binds to TGF-β. In some embodiments, the binding pair member is selected from the group consisting of Lyp-1, RGD-4C, NGR and F-3. Also in some embodiments, the protein that specifically binds to TGF-β is selected from the group consisting of TGF-β receptor I, TGF-β receptor II and soluble TGF-β receptor II.

An aspect of the disclosure related to all of the above-described methods is the use of an adenovirus expressing an E1A protein deficient in facilitating host cell transition to the S phase of growth and further comprising a coding region for a protein that specifically binds to TGF-β in the preparation of a medicament for the treatment of cancer.

Yet another aspect of the disclosure is drawn to an adenovirus expressing an E1A protein deficient in facilitating host cell transition to the S phase of growth and further comprising a coding region for a protein that specifically binds to TGF-β. Any of the adenoviruses described herein, such as the adenoviruses described above in the context of describing the methods of the disclosure, is contemplated as being embraced within this aspect of the disclosure.

A related aspect of the disclosure is directed to a pharmaceutical composition comprising any of the adenoviruses described herein, including the above-described adenoviruses, and a pharmaceutically acceptable diluent, carrier or excipient. Any one of skill in the art would know whether a diluent, carrier or excipient were pharmaceutically acceptable or would know how to determine it using routine procedures. Any diluent, carrier or excipient known in the art is contemplated for use in this aspect of the disclosure.

Another related aspect of the disclosure is drawn to a kit comprising any of the pharmaceutical compositions described herein, including the compositions described above.

Other features and advantages of the subject matter of the disclosure will become apparent from the following detailed description. It should be understood, however, that the detailed description and the specific examples, while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWING

FIG. 1. Schematic diagram of adenoviral constructs. The gene for the sTGFβRII (amino acids 1-159) under the control of CMV promoter was inserted in the E3 region in rAd-sTRII adenoviral vector and in the E1 region in Ad-s-TRII adenoviral vector. rAd-sTRII has two small deletions (⋄⋄) 4 to 25 and 111 to 123 amino acids in E1A protein (dl01/07) and RID-α, β, and 14.7K protein deletions in E3 region. Ad-sTRII is an E1, E3-deleted adenovirus. The maps were not drawn to scale.

FIG. 2. A. Schematic diagram depicting key features of the E1A region having the dl01/07 structure. The upper drawing shows the structure of the E1A region of wild-type adenovirus 5. It contains 289 amino acids (aa) and encodes proteins, which bind to the p300 and Rb proteins, the regulators of cell cycle progression. The lower drawing shows the EIA structure of the dl01/07 mutant virus. The dl01/07 virus has two deletions. One deletion is aa 4-25 (dl01), and the second deletion is aa 111-123 (dl07). Because of these mutations, the E1A region of dl01/07 is shorter (254 aa). In dl01/07, the resultant E1 proteins cannot bind with p300 or Rb proteins. Thus, dl01/07 is ineffective for S-phase progression in primary cells and cannot replicate. However, cancer cells are able to progress to S-phase, permitting virus replication in these cells. B. Schematic diagram of sTGFβRIIFc cDNA. The upper panel shows the TGFβRII structure. The structure contains three domains—an extracellular domain (amino acids 1-159), a transmembrane domain, and a serine/threonine kinase domain. The lower panel shows the extracellular domain fused to human IgG1Fc (Fc). The resulting cDNA sTGFβRIIFc was cloned in an oncolytic adenovirus backbone dl01/07.

FIG. 3. Expression of sTGFβRII from MDA-MB-231 cells. A, MDA-MB-231 cells were incubated with replication-deficient AdNull or Ad-sTRII or oncolytic rAd-sTRII (100 MOI) for 3 hours and washed, and DMEM without serum was added to the cells and incubated for 24 hours. B and C, MDA-MB-231 cells were incubated with different dosages of Ad-sTRII or rAd-sTRII (0, 1, 5, 25, 100, and 200 MOI) for 3 hours and washed and DMEM without serum was added to the cells and incubated for 24 hours. Culture media and cells were collected separately for Western blot analysis using antibodies against TGFβRII. The protein loading in each lane from cell lysates was determined by probing β-actin. MW, molecular weight.

FIG. 4. Deglycosylation of sTGFβRII. Culture media from Ad-sTRII- or rAd-sTRII-infected MDA-MD-231 cells were treated with PNGase F (+) or left untreated (−). The proteins were subjected to SDS-PAGE and analyzed by Western blot for sTGFβRII using antibody against TGFβRII.

FIG. 5. Binding of TGF-β and inhibition of p38 MAPK phosphorylation by sTGFβRII. A, culture media from uninfected or infected MDA-MB-231 cells were incubated with recombinant TGF-β1 protein (40 ng) and mixed with wheat germ agglutinin-Sepharose beads. After extensive washing, the beads were subjected to SDS-PAGE (15%) and analyzed by Western blot using an antibody against TGF-β1. A recombinant TGF-β1 protein was included as positive control. B, serum-starved MDA-MB-231 cells were stimulated with TGF-β1 for various times (0, 10, 20, and 30 minutes and 1, 2, 3, and 4 hours). C. serum-starved MDA-MB-231 cells were treated for 1 hour with culture media obtained from AdNull-, Ad-sTRII-, or rAd-sTRII-infected MDA-MB-231 cells. Culture media from MDA-MB-231 cells without Ad infection served as control. Amount of phosphor-p38 and total p38 was analyzed by Western blot using antibodies specific for phosphorylated and total p38 MAPK.

FIG. 6. Cytotoxicity of recombinant adenoviruses to breast cancer cells. Effect of different adenoviruses on cell growth was assayed by plating breast cancer cells (500 per well) in triplicate in 96-well plates. Cells were infected with AdNull, Ad-sTRII, rAd-TK, or rAd-sTRII for 7 days and stained as described herein. A, MDA-MB-231 cells. B, MCF-7 cells. C, IC₅₀ ratio between Ad-sTRII and rAd-STRII was calculated for different breast cancer cell lines. Points, means of three separate experiments, each conducted in triplicate; bars, SE.

FIG. 7. Replication of adenoviruses in MDA-MB-231 cells. MDA-MB-231 cells were infected with different viruses for 3 and 48 hours. Both media and cells were recovered and processed for the release of viruses. Viral titer was determined on HEK-293 cells by plaque assay. Columns, mean of three separate experiments, each done in duplicates; bars, SE. Inset, the fold increase in viral titers from 3 to 48 hour incubation.

FIG. 8. A. Construction of Ad.sTBRFc. Ad.sTBRFc was constructed using an homologous recombination method as described herein, and purified by double cesium chloride gradient ultracentrifugation as described (Katayose et al., 1995; Craig et al., 1997). B. The schematic structure of Ad.sTβRFc. The key regions of recombinant Ad.sTβRFc shown are: a mutant E1A region (01/07), the expression cassette containing sTGFβRIIFc inserted at the E3 site of the adenovirus genome (the part of E3 encoding the adenovirus death protein is left intact), EIB, L1-L5, and E4 (adenoviral E2 sequences are not shown).

FIG. 9. A. Ad.sTβRFc-mediated expression of sTGFβRIIFc protein in breast cancer cells. MDA-MB-231 cells (Left Panel) and MCF-7 (Right Panel) cells were plated in 6-well plates (2×10⁵ cells/well). The next day, cells were infected with 100 pfu/cell of Ad.sTβRFc or Ad.Null. Both cells and media were collected and subjected to Western blot analysis. Blots were probed with anti-TGFβRII antibody (Santa Cruz Biotechnology, Santa Cruz, Calif.) or anti-actin (Santa Cruz), and developed by using the enhanced chemiluminescence technique (Amersham). The left side shows molecular weight markers. B. TGFβ binding with conditioned media containing sTGFβRIIFc. Conditioned media derived from Ad.sTβRFc-infected MDA-MB-231 cells was incubated with TGFβ-1. The TGFβ-TGFβRIIFc complexes were bound with WGA and subjected to Western blot analysis by probing with rabbit anti-TGFβ-1 polyclonal antibody (Promega). Lane 1 received pure TGFβ-1 as a positive control. Note that the TGFβ-1 band is present when the media from Ad.sTβRFc-infected cells was used (lane 2), but not from the Ad.TK-infected cells (Lane 3). C. Inhibition of TGFβ transcriptional activity. MVILu cells (5×10⁵ per well) were transfected with 2 μg firefly luciferase reporter plasmid p3TP-lux and 0.2 μg Renilla luciferase plasmid pRL-TK using lipofectamine 2000 (Invitrogen). The next day, cells were placed in serum-free medium and serum-starved for 24 hours, followed by incubation in conditioned media derived from control Ad.TK- or Ad.sTβRFc-infected (24 hours infection) MDA-MB-231 cells in the absence or presence of TGFβ-1 (2 ng/ml) for 24 hours. Proteins were extracted and luciferase activity was measured in cell lysates using the dual luciferase assay kit. Bioluminescence was measured using a Turner Designs Luminometer. Results shown are luciferase activities when the media from control, Ad.TK, or Ad.sTβRFc infected MDA-MB-231 cells were used. p<0.05 versus the basal level luciferase activity are indicated by “*”. D. Viral production assay. MDA-MB-231 cells (10⁵) were plated in six-well plates, and infected with Ad(E1-).Null, Ad.sTβRFc, or dl309 (100 pfu/cell) and the viral titers were determined by plaque assays as described herein. Viral plaques were counted following an 8-day incubation, and are shown as plaque forming units. Results shown are the average of three determinations (±SE).

FIG. 10. A. In vivo evaluation of Ad.sTβRFc administration on MDA-MB-231 tumor xenografts in nude mice. MDA-MB-231 tumors were established subcutaneously in nude mice and treated with oncolytic viruses (Ad.sTβRFc, Ad.TK), sTGFβRIIFc, or buffer as described herein. Tumor volumes were measured and calculated using the formula (a×b²)×0.52. There were eight animals in each group. Compared to buffer group p, the value for Ad.sTβRFc group is <0.0001, for Ad.TK group is <0.0001, and for sTGFβRIIFc group is 0.16 (day 57). B. Percentage of tumor-bearing mice in various groups of animals (control, Ad.sTβRFc, Ad.TK, and sTGFβRIIFc treated groups). Animals (from FIG. 4A) were monitored weekly for the presence or absence of tumors. The percentage of animals with tumors is shown.

FIG. 11. A. Additional in vivo evaluation of intravenously administered Ad.sTβRFc on MDA-MB-231 tumor xenografts in nude mice. MDA-MB-231 tumors were established subcutaneously in nude mice and treated with oncolytic Ad.sTβRFc virus or buffer as described herein. Tumor volumes were measured and calculated using the formula (a×b²)×0.52. Following injection of oncolytic virus or buffer control into nude mice bearing MDA-MB-231 breast tumor xenografts, tumor volumes were monitored weekly. Tumor volumes as a function of time were plotted, with the oncolytic Ad.sTβRFc virus tumor volumes indicated by triangles and the tumor volumes of the buffer control group shown by squares.

### DETAILED DESCRIPTION

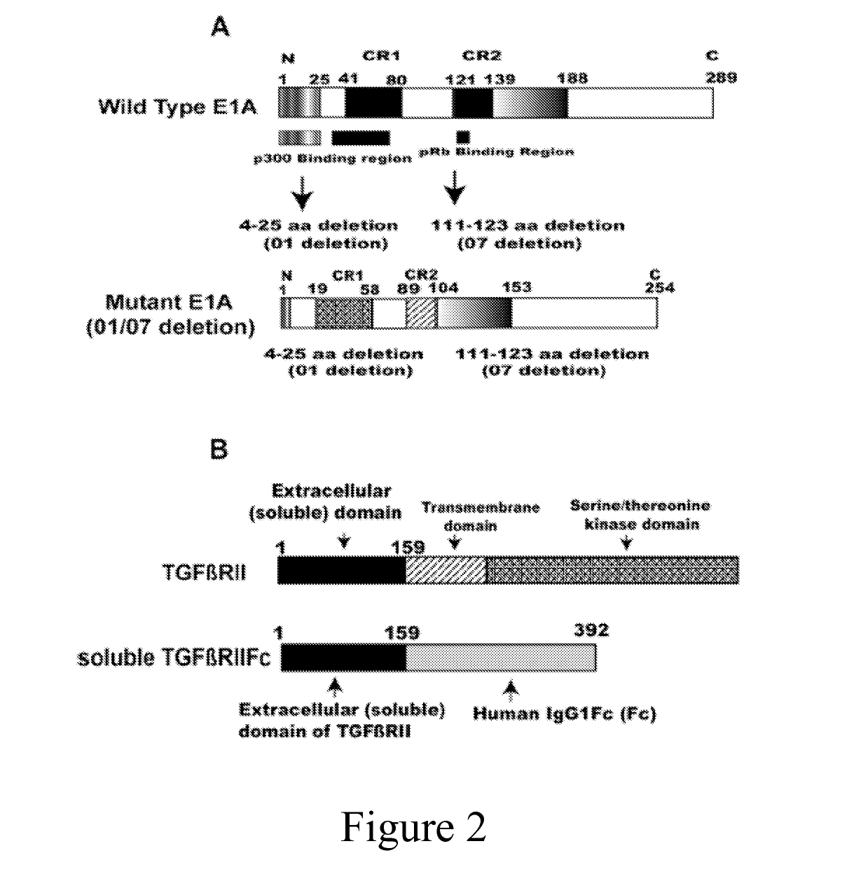

Disclosed herein are recombinant oncolytic adenoviruses useful as anti-tumor agents. In preferred embodiments, an adenovirus mutant dl01/07 is used because it can replicate in all cancer cells regardless of their genetic background (Howe et al., 1990; Howe et al., 2000). The dl01/07 virus has two deletions in E1A region, one deletion is 4 to 25 amino acids (dl01), and the second deletion is 111 to 123 amino acids (dl07). The resultant E1A proteins cannot bind with p300/CBP or pRb proteins (8). Therefore, in primary cells, dl01/07 is ineffective for S-phase induction, and the adenovirus cannot replicate (FIG. 2A). However, cancer cells are able to progress to S phase, thus permitting virus replication in these cells. Since most human tumors are heterogeneous and have varied genetic backgrounds, while remaining susceptible to infection with human adenoviruses (Seth et al., 1996; Rakkar et al., 1998), recombinant oncolytic adenoviruses based on the dl01/07 mutant are expected to be useful anti-cancer therapeutics.

To further augment the effectiveness of the dl01/07 adenovirus as an anti-tumor agent, the adenovirus was armed with a soluble form of TGFβ Receptor-II, optionally fused to a stabilizing peptide such as immunoglobulin fused to Fc (sTGFβRIIFc). The sTGFβRIIFc protein binds to TGFβ-1 and inhibits TGFβ signaling in target cells (Yang et al., 2002). The TGFβ pathway was targeted because high levels of TGFβ-1 have been shown to have tumor growth-promoting activities by enhancing angiogenesis, invasion, and metastasis, and by inhibiting immune functions (Inge et al., 1992; Yin et al., 1999; Akhurst and Derynck, 2001; Derynck et al., 2001; Hiraga et al., 2001; Teicher, 2001; Iwasaki et al., 2002; Wakefield and Roberts, 2002; Zhao et al., 2002; Guise and Chirgwin, 2003; Roberts and Wakefield, 2003; Tang et al., 2003; Iyer et al., 2005; Thomas and Massague, 2005). Inhibition of excessive TGFβ signaling activity in turn inhibits EMT conversion, tumor invasion and metastasis, angiogenesis, and osteolysis, as well as reversing immunosuppression.

An oncolytic adenovirus armed with sTGFβRII (rAdsTRII) was constructed by inserting the soluble 159-amino-acid residue domain of the TGFβRII into the dl01/07 adenoviral genome. As a control for viral replication, a replication-deficient adenovirus containing sTGFβRII (Ad-sTRII) and a replication-competent dl01/07 expressing herpes simplex virus thymidine kinase (rAd-TK) were also constructed. These constructs were used to investigate whether adenoviral vector-mediated expression of sTGFβRII in the extracellular environment could bind to TGF-β, resulting in inhibition of TGF-β signaling in target cells.

There was an initial concern that adenoviral-mediated expression of sTGFβRII could potentially interfere with viral replication. Therefore, the effect of sTGFβRII expression on adenoviral replication was investigated in breast cancer cells. The cytotoxicity and replication potential of rAd-sTRII and Ad-sTRII were assessed in breast tumor cells. The results indicate that whereas the infection of breast tumor cells with rAd-sTRII and Ad-sTRII produced functional sTGFβRII protein, only rAd-sTRII replicated in tumor cells. Thus, it is possible to simultaneously achieve adenoviral replication and expression of the secreted form of a functional sTGFβRII protein, indicating that rAd-sTRII are useful in cancer therapy.

In recent years, replication-competent oncolytic adenoviral vectors as potential antitumor agents have been developed. To augment the anti-cancer effects of replicating adenoviruses, oncolytic adenoviruses could be armed with other genes, such as suicide genes, in a manner similar to the extensive development of the recombinant replication-deficient adenoviruses (1-4, 37). Data disclosed herein establish that the sTGFβRII gene was successfully inserted into the genomes of a replication-competent adenovirus and a control replication-deficient adenovirus in vitro evaluations in breast tumor cells demonstrated the promise of the compositions as anti-cancer therapeutics. In addition, the data establish that genes encoding fusions of sTGFβRII and a coding region for a stabilizing peptide, such as the F_{C} region of an immunoglobulin, also show promise as anti-cancer therapeutics, based on in vitro and in vivo data.

As disclosed hereinbelow, sTGFβRII was overexpressed in breast cancer cells after infection with an rAd-sTRII adenoviral vector. Vector-mediated expression of sTGFβRII was dependent on viral dose. Western blot analyses of the infected cells indicated multiple size protein bands. However, the multiple protein bands were not due to degradation product(s) of sTGFβRII, but due to the glycosylation of sTGFβRII, as the treatment of the secreted proteins with Peptidyl-N-glycosidase F (PNGase F) converted the various heterogeneous bands into two distinct protein bands. More importantly, the secreted sTGFβRII protein was shown to bind to TGF-β1 and inhibited TGF-β-stimulated p38 MAPK in target cells, indicating that sTGFβRII was fully functional. Similar levels of sTGFβRII functional proteins were produced by replication-deficient and replication-competent adenoviruses, establishing that viral replication had no adverse effect on expression of sTGFβRII protein.

Overexpression of TGF-β ligands has been reported in many tumor types and elevated levels of TGF-β in tumor tissues correlate with markers of a more metastatic phenotype and/or with poor patient outcome (38, 39). Based on in vitro data disclosed herein that rAd-sTRII-mediated expression and secretion of sTGFβRII into the extracellular environment inhibits TGF-β signaling, the administration of rAd-sTRII in vivo is expected to produce sTGFβRII that will be systemically released into the blood. This release will inactivate the “overactive” TGF-β signaling associated with breast cancers and will result in the inhibition of tumor invasion and metastasis.

Data disclosed herein show that the rAd-sTRII is fully replication competent compared with the phenotypically wild-type adenovirus dl309. Given the tight interaction of the cellular machinery with adenoviral replication, one concern was that heterologous protein expression could potentially interfere with adenoviral replication, diminishing or defeating the therapeutic purpose of administering replicating adenoviral vectors. Because conditionally replicating viruses in conventional use often exploit differences in cell cycle status, programmed cell death, and cellular DNA synthesis between normal and tumor cells, the heterologous protein could potentially interact with these cellular pathways/machinery and interfere with adenoviral replication, even in the tumor cells. Examples of such proteins are the regulators of cell cycle (p16^{INK4A} and p21^{WAF1/Cip1}) apoptosis (wild-type p53 and Bax), and DNA and protein synthesis (suicide gene plus a pro-drug). Given the multiple pathways involved in TGF-β-mediated signaling, there was a possibility that interfering with TGF-β pathways would interfere with adenoviral replication. In this regard, it is a significant finding that overexpression of vector-derived sTGFβRII does not compromise adenoviral replication in breast tumor cells. However, it was surprising that sTGFβRII overexpression did not enhance the cytotoxic effect of the oncolytic virus.

Another point worth noting is the choice of replicating adenoviral mutant to overexpress the transgene of interest. Exemplified herein is the use of the dl01/07 adenovirus backbone, because dl01/07 expresses a mutant E1A gene product defective in binding both p300/CBP and pRb. pRb and p300 regulate the activity of E2F, which activates genes involved in the transition from the G₁ phase to the S phase of the cell cycle. All tumor cells exhibit uncontrolled cell growth due to a deregulated G₁-S phase transition of the cell cycle. In cycling tumor cells, E2F is constitutively active because of disruption in the pRb/P16^{INK4a} cyclin D pathway, including E2F-1 gene amplification. Therefore, dl01/07 can replicate in tumor cells regardless of their genetic background (40), making it an attractive vector for treating a variety of cancers.

Thus, rAd-sTRII replication in infected cells and the simultaneous production and release of sTGFβRII in the extracellular medium, resulting in the inhibition of TGF-β signaling in the target cells, provides a powerful tool to simultaneously treat both primary tumors and metastases in breast cancer. Although these studies have focused on breast cancer cells as a target, it is expected that modified adenoviral vectors expressing a binding partner for a protein associated with tumorigenesis, such as rAd-sTRII, will find applications in targeting many cancers, especially those malignancies in which the expression level of a cancer- or tumor-associated protein is correlated with oncogenesis or tumorigenesis, such as the correlation of TGF-β overexpression with tumorigenesis enhancement.

A dl01/07-based oncolytic adenovirus expressing sTGFβRIIFc (Ad.sTβRFc) was also constructed and characterized in various in vitro and in vivo assays. The infection of breast cancer cells with Ad.sTβRFc produced high levels of sTGFβRIIFc that were subsequently released into the extracellular space. The sTGFβRIIFc produced in the medium was shown to bind to TGFβ-1 and to inhibit TGFβ signaling in target cells. The infection of breast cancer cells also produced high levels of viral titers. Direct injection of Ad.sTβRFc into human xenografts established in nude mice caused significant inhibition of tumor growth, resulting in tumor regressions in more than 85% of the animals. These results indicate that Ad.sTβRFc is useful as a therapeutic for breast cancer therapy.

Beyond the use of adenoviruses deficient in E1A protein, such as Ad dl01/07, which naturally target cell cycle dysregulated cells (cancer cells), an adenovirus containing a tumor-specific promoter used to control expression of an essential gene, such as a gene required for viral replication, would also provide a viral vector suitable for use in cancer therapy. Exemplary tumor-specific promoters include hTERT promoters, such as modified hTERT promoters, and a promoter for a small mucin-like protein. In addition, viral vector targeting is achievable by fusing a binding pair member to an adenoviral protein involved in cell contact during the infection process. For example, the adenoviral fiber protein, emanating from a penton base in the capsid, is a suitable adenoviral protein for fusion to a binding pair member, such as Lyp-1, RGD-4C, NGR, F-3, or any binding pair member having a partner preferentially associated with a cancer, e.g., tumor, cell.

The following examples are included to demonstrate embodiments of the disclosed subject matter. Those of skill in the art will, in light of the present disclosure, appreciate that changes can be made in the specific embodiments which are disclosed and still obtain a like or similar result without departing from the spirit and scope of the disclosed subject matter.

### Example 1

### Cell Culture

HEK-293 (ATCC CRL-1573), SK-BR-3, MDA-MB-468, MDA-MB-453, T47D, MCF-7, and MDA-MB-231 (source ATCC) were cultured in Dulbecco's Modified Eagle's Medium (DMEM) (Mediatech, Inc., Herndon, Va.) containing 10% fetal bovine serum (FBS; Mediatech, Inc.) and 1% penicillin/streptomycin (Invitrogen, Carlsbad, Calif.). MV1Lu, MCF-7, (source ATCC) were grown in EMEM (ATCC) containing 10% FBS and 1% penicillin/streptomycin (Invitrogen).

### Example 2

### Adenoviral Constructs

For construction of replication-deficient adenovirus Ad-sTRII, an approximately 0.5-kb NotI-HindIII DNA fragment encoding codons 1-159 of the TGFβRII gene from pBS-SK(−)/sTRII (24) was cloned in the NotI and HindIII sites of pShuttle-CMV (Stratagene, Inc.). The resulting shuttle vector, pShuttle-CMV/sTRII, was then recombined in E. coli BJ5183 by homologous recombination with the E1- and E3-deleted pAdEasy-1 adenoviral backbone vector (Stratagene, La Jolla, Calif.) to generate a packageable adenoviral genome, pAd-sTRII (25). The Ad-sTRII vector was produced by transfecting PacI-digested pAd-sTRII into HEK-293 cells using LipofectAMINE 2000 (Invitrogen). For construction of replication-competent adenovirus rAd-sTRII, an approximately 550-bp XbaI fragment from pShuttle-CMV/sTRII encoding codons 1-159 of the TGFβRII gene was cloned into XbaI-cut plasmid p309-CMV-poly(A) to produce the shuttle vector p309/sTRII. The 11-kb PacI-AscI fragment from p309/sTRII was recombined with BstBI- and SpeI-cut adenoviral backbone plasmid pTG07-4609 in E. coli BJ5183 to produce adenoviral genome plasmid pTG07-4609/sTRII. Adenovirus rAd-sTRII was generated by transfecting PacI-cut pTG07-4609/sTRII into HEK-293 cells. rAd-TK was constructed by similar procedures except that the herpes simplex virus thymidine kinase (HSV-TK) gene was inserted instead of the sTGFβRII gene. E1-deleted, replication-deficient adenovirus devoid of any foreign cDNA (AdNull) is known in the art and has been previously described (26). dl309 is a phenotypically wild-type adenovirus (27). Adenoviruses were amplified in HEK-293 cells and purified by cesium chloride gradient ultracentrifugation, and the titers were calculated using published conventional methods (28, 29, incorporated herein by reference).

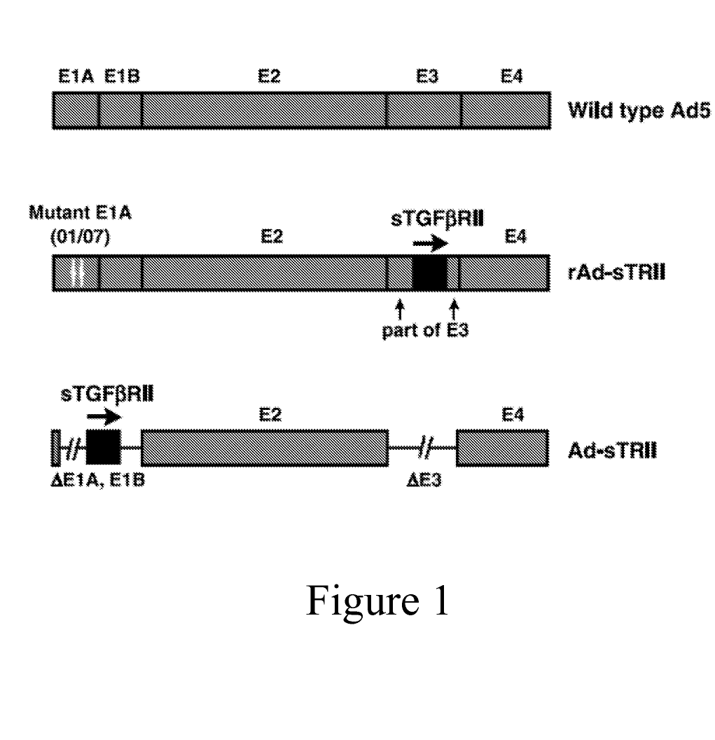

To generate Ad-sTRII and rAd-sTRII, the cDNA encoding the complete extracellular domain of human TGFβRII (amino acid residues 1-159) under the control of cytomegalovirus promoter (CMV) was placed in their individual genomes. The schematic diagram of the structure of adenoviruses is shown in FIG. 1. It should be noted that the replication-deficient Ad-sTRII vector has an E1 deletion, whereas rAd-sTRII is a conditionally replicating adenovirus due to two short deletions in the E1A gene (dl01/07; FIG. 1; ref. 8).

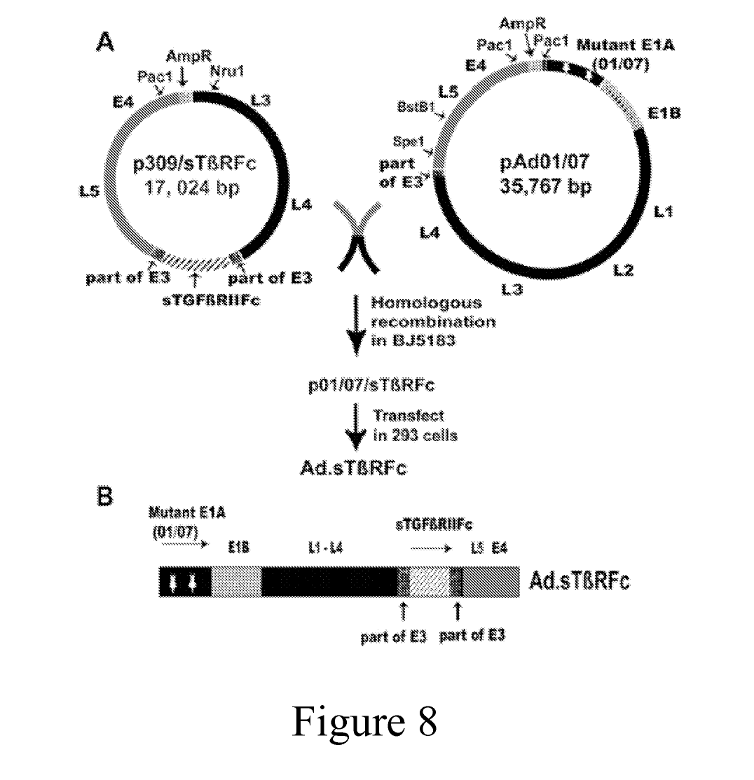

sTGFβRIIFc cDNA (FIG. 1B), driven by a CMV promoter was cloned into a shuttle vector, permitting insertion of the cDNA at the E3 site of d/01/07. Ad.sTβRFc was generated by homologous recombination of the shuttle vector with an adenoviral plasmid containing the 01/07 E1A gene (FIG. 8A). The schematic structure of Ad.sTβRFc is shown in FIG. 8B. In particular, a 1.2 kb HindIII-ApaI fragment from pcDNA3/SR2F (Yang et al., 2002) containing cDNA encoding the soluble form of TGFβ Receptor-II fused to human IgG Fc γ was first cloned in HindIII- and ApaI-digested pBS-SK(+). The sTGFβRIIFc cDNA was then cloned into plasmid p309 to produce the shuttle vector p309/sTβRFc. The 15.7 kb PacI-NruI fragment from p309/sTβRFc was then co-transformed into E. coli BJ5183 with the BstBI- and SpeI-cut adenoviral backbone plasmid pAd01/07. Homologous recombination of the shuttle vector and plasmid pAd01/07 produced adenoviral genome plasmid p01/07/sTβRFc. PacI-cut p01/07/sTβRFc was transfected into HEK-293 cells to produce the recombinant adenovirus Ad.sTβRFc. Adenovirus was collected after 10 days, amplified in HEK-293 cells, and purified by double cesium chloride gradient ultracentrifugation, as described (Katayose et al., 1995; Craig et al., 1997).

### Example 3

### Expression of sTGFβRII and Derivatives Thereof in Tumor Cells

MDA-MB-231 cells (1×10⁶ per well in a six-well plate) were plated in DMEM containing 10% FBS and incubated at 37° C. overnight. The next morning, cells were infected with 100 plaque-forming units/cell (unless otherwise mentioned) of adenovirus for 3 hours. Cells were washed and incubated with DMEM without FBS for 24 hours. Medium and cells were separately dissolved in SDS sample buffer and subjected to Western blot analysis as previously described (28, 30). Blots were probed with antibody reactive against TGFβRII (H-567; Santa Cruz Biotechnology, Santa Cruz, Calif.) or actin protein (1-19; Santa Cruz Biotechnology).

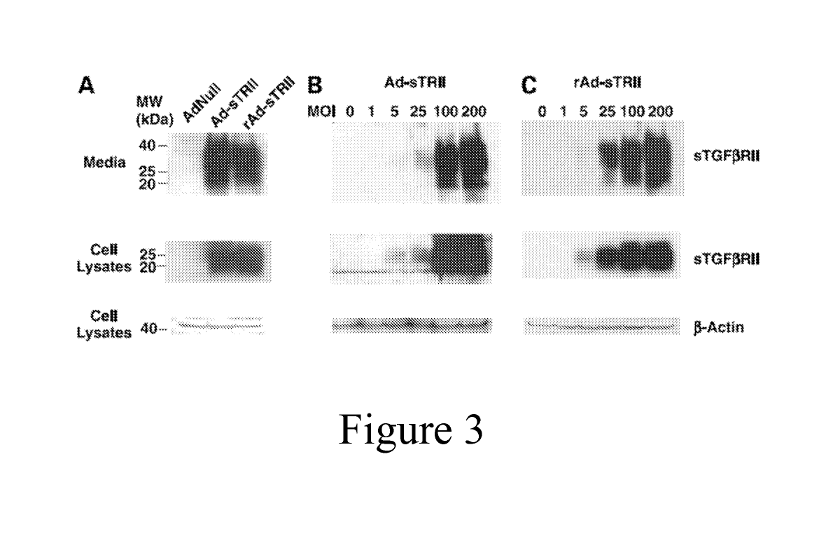

MDA-MB-231 breast cancer cells were exposed to AdNull, Ad-sTRII, or rAd-sTRII for 24 hours and subjected to Western blot analyses. As shown in FIG. 3A, there were no detectable protein bands reactive with antibody against TGFβRII in cells infected with AdNull. In contrast, strong protein bands appeared in both Ad-sTRII- and rAd-sTRII-infected cells. In cell lysates, there were protein bands with molecular weights ranging from 20 to 25 kDa, whereas in cell media, protein bands shifted to the higher position with molecular weights ranging from 20 to 40 kDa. FIGS. 3B and C shows the dose-dependent increases of sTGFβRII expression in both media and cell lysates. Quantitation revealed that cell lysates contained 74.2±3.7 μg/mg protein (MDA-231 cells) and 31.4±5.1 μg/mg protein (MCF-7 cells); extracellular media contained 4.5±0.17 μg/ml medium (MDA-231 cells) and 3.9±0.63 μg/ml medium (MCF-7 cells). Infection of other breast tumor cells with these viruses also resulted in the overexpression of sTGFβRII.

To investigate Ad.sTβRFc-mediated expression of sTGFβRIIFc protein, MDA-MB-231 and MCF-7 breast tumor cells were infected with Ad.sTβRFc (100 pfu/cell) for 48 hours. Both media and cells were subjected to Western blot analysis and probed with anti-TGFβRII. Cells were plated (2×10⁵ cells per well in 6-well plates) in medium containing 10% FBS and incubated at 37° C. overnight. The next morning, cells were infected with 100 adenoviral plaque forming units (pfu)/cell for 48 hours. Media and cells were separately dissolved in SDS sample buffer, and subjected to Western blot analyses as previously described (Katayose et al., 1995; Craig et al., 1997). Blots were probed with antibody reactive against either TGFβRII (Santa Cruz Biotechnology, Santa Cruz, Calif.) or actin (Santa Cruz Biotechnology).

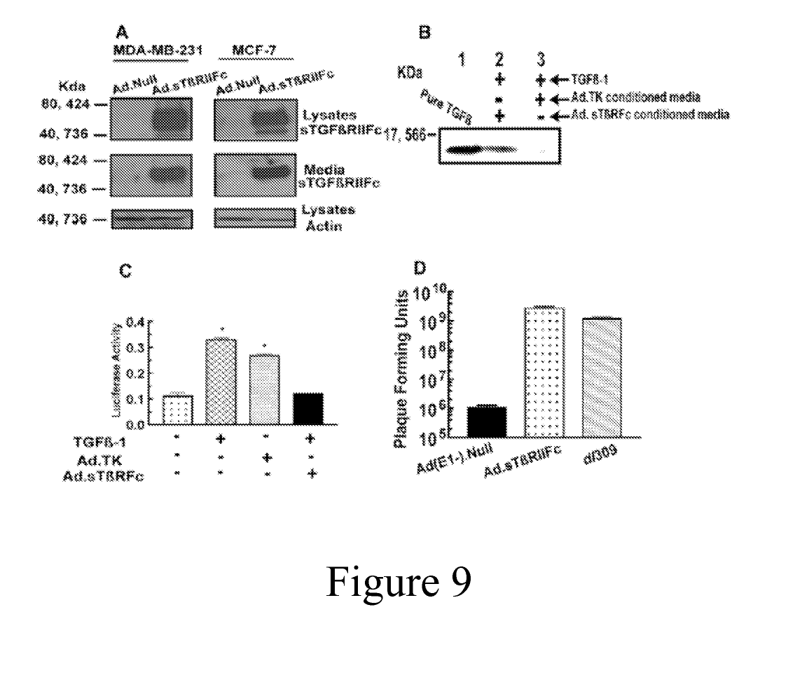

The cells infected with Ad.sTβRFc, as well as the extracellular media, showed a diffuse protein band of 50-70 Kd (FIG. 9A). These results indicate that the infection of breast cancer cells with Ad.sTβRFc results in the expression of sTGFβRIIFc, which is subsequently secreted out of the cells.

### Example 4

### Treatment of sTGFβRII with Peptidyl-N-Glycosylase F (PNGase F)

Sixty microliters of culture media from Ad-sTRII- or rAd-sTRII-infected MDA-MB-231 cells were denatured at 100° C. for 10 minutes and treated with PNGase F (New England Biolabs, Beverly, Mass.) for 2 hours at 37° C. according to manufacturer's instructions. The proteins were subjected to SDS-PAGE and analyzed by Western blot using rabbit anti-TGFβRII polyclonal antibody.

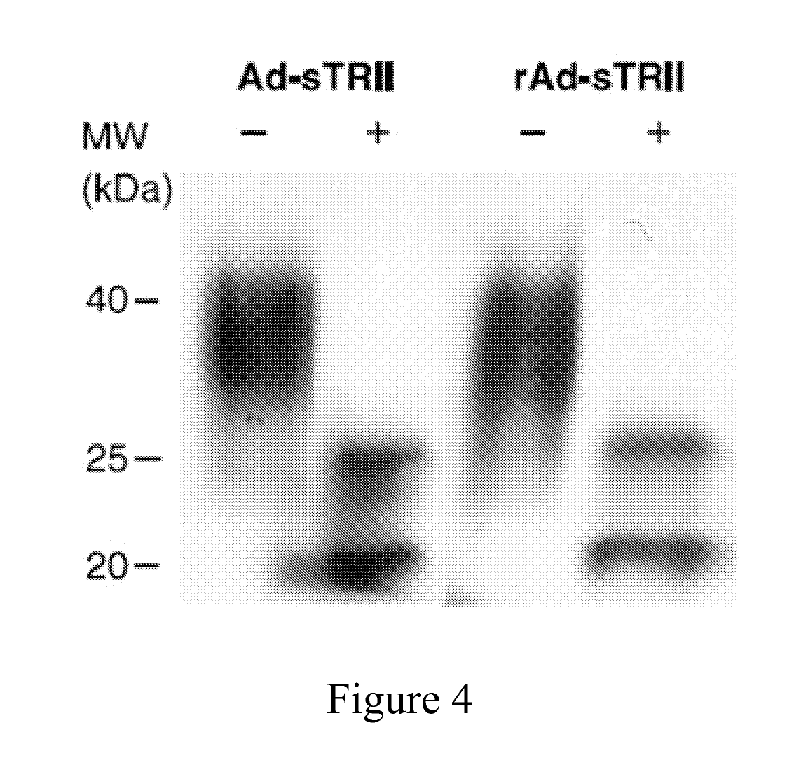

The predicted molecular mass of truncated TGFβRII (amino acid residues 1-159) is about 18 kDa. After cleavage of the hydrophobic leader sequence, the length of this truncated receptor is 136 amino acid residues, and the predicted molecular mass is about 15.5 kDa. However, as described above, the cells did not produce a distinct protein band upon gel electrophoresis, instead producing a smear of high molecular weight. The secreted soluble receptor contains complex N-linked oligosaccharides as well as additional sialic acid residues (31). The secreted sTGFβRII receptor from both Ad-sTRII- and rAd-sTRII-infected cells were treated with N-glycosidase F (PNGase F), an amidase that cleaves between the innermost GlcNAc and asparagine residues of high-marmose, hybrid, and complex oligosaccharides from N-linked glycoproteins (32). The protein smear was resolved into two major distinct bands (about 25 and about 20 kDa), indicating that the sTGFβRII produced by MDA-MB-231 cells is a heterogeneously glycosylated protein (FIG. 4).

### Example 5

### sTGFβRII Forms Bind to TGFβ-1 and Inhibit TGFβ Signaling

Vector-mediated sTGFβRIIFc contributes to the benefits provided by the products and methods described herein by binding to the TGFβ protein, causing the inhibition of TGFβ signaling in target cells. To examine the binding of TGFβ by sTGFβRII, MDA-MB-231 cells (1×10⁶ per well in six-well plates) were uninfected or infected with different adenoviruses at a multiplicity of infection (MOI) of 100 for 3 hours in growth medium. Cells were washed and incubated in 1.7 mL serum-free DMEM medium for 20 hours. The culture media were collected, and 200 μL of culture media were mixed with TGF-β1 (40 ng; Sigma, St. Louis, Mo.) for 1 hour at 4° C.; 50 μL of wheat germ agglutinin-Sepharose agarose beads (Vector Laboratories, Burlingame, Calif.) were then added and incubated for 1 hour at 4° C. The beads were washed six times with buffer (50 mmol/L NaCl, 10 mmol/L Tris-HCl, 5 mmol/L EDTA, 1% Triton X-100 (pH 7.4)) and subjected to SDS-PAGE (15%). The proteins were transferred onto Immun-Blot polyvinylidene difluoride membranes (Bio-Rad, Hercules, Calif.) and probed with rabbit anti-TGF-β1 polyclonal antibody (Promega, Madison, Wis.).

To examine the binding of TGFβ with sTGFβRIIFc, pure recombinant TGFβ-1 was incubated with culture media derived from Ad.sTβRFc-infected MDA-MB-231 cells. The mixture was combined with wheat germ agglutinin-Sepharose (WGA) beads, which bind glycosylated proteins, including sTGFβRIIFc. The complexes were analyzed by Western blots using anti-TGFβ-1. MDA-MB-231 cells (1×10⁶ per well in six-well plates) were uninfected or infected with different adenoviruses (100 pfu/cell) for 3 hours in growth medium. Cells were washed and incubated in 1.7 ml serum-free DMEM medium for 20 hours. The culture media were collected and 200 μl of culture media were mixed with TGFβ-1 (40 ng) (Sigma) for 1 hour at 4° C.; wheat germ agglutininsepharose agarose beads (Vector Laboratories) were then added and incubation was continued for 1 hour at 4° C. The beads were then washed six times with buffer (50 mM NaCl, 10 mM Tris-Cl, 5 mM EDTA, 1% Triton X-100, pH 7.4) and subjected to SDS-PAGE (15%). The proteins were transferred onto Immun-Blot™ PVDF membranes (Bio-Rad) using conventional techniques and probed with rabbit anti-TGFβ-1 polyclonal antibody (Promega).

FIG. 9B shows the presence of TGFβ-1 in the precipitate when the medium from Ad.sTβRFc infected cells was used. However, TGFβ-1 was not present when the medium from Ad.TK-infected cells was used (Ad.TK is a 01/07 based oncolytic virus expressing HSV-TK gene. These results indicate that the conditioned media containing the secreted soluble TGFβRIIFc binds to TGFβ-1.

To assess whether Ad.sTβRFc-mediated expression of sTGFβRIIFc was capable of blocking TGFβ functions, the inhibition of a TGFβ-responsive luciferase reporter gene was examined. Plasminogen activator inhibitor-1 (PAI-1) is a known target gene of TGFβ signaling. TGFβ-mediated transcriptional activity was assessed in mink lung epithelial MV1Lu cells known to be TGFβ sensitive (Kanamoto et al., 2002). Plasmid p3TP-lux (Wrana et al., 1992), containing multiple response elements from the promoter of plasminogen activator inhibitor, was used in a reporter assay. MV1Lu cells were transiently transfected with p3TP-lux reporter plasmid (with and without TGFβ-1), and luciferase activity was measured. MV1Lu cells (1×10⁶ per well) were transfected with 2 μg firefly luciferase reporter plasmid p3TP-Lux and 0.2 ug Renilla luciferase plasmid pRL-TK using Lipofectamine 2000 according to the manufacturer's protocol (Invitrogen). The next day, cells were placed in serum free medium and serum-starved for 24 hours and then incubated with various conditioned media in the absence or presence of TGFβ-1 (2 ng/ml) for 24 hours. Proteins were extracted using 1× Passive Lysis Buffer (Promega, Madison, Wis.). Luciferase activity was then measured in cell lysates using the dual luciferase assay kit (E-1910). Bioluminescence was measured using a Turner Designs luminometer (TD-20/20).

As shown in FIG. 9C, in the absence of TGFβ-1, a basal level of luciferase activity was detected. However, addition of TGFβ-1 resulted in an increase in the luciferase activity of about 3-fold. The co-incubation of media from Ad.TK-infected cells with TGFβ-1 did not inhibit the TGFB-1-dependent increase in luciferase activity. However, the TGFβ-1-dependent luciferase activity was significantly decreased when culture media from Ad.sTβRFc-infected cells were used, indicating that the media containing sTGFβRIIFc inhibited TGFβ-1 transcriptional activity (FIG. 9C).

### Example 6

### Functional Assays

### p38 MAPK Activation in MDA-MB-231 Cells by TGF-β1

MDA-MB-231 cells grown in normal growth medium were serum-starved overnight in DMEM without FBS, washed, and incubated in fresh DMEM without FBS. TGF-β1 (5 ng/mL) was added to the cells and incubated for 0, 10, 20, 30, 60, 120, 180, and 240 minutes at 37° C. Total cell lysates were subjected to Western blot analyses using antibodies against phospho-p38 (sc-7975-R, Santa Cruz Biotechnology) or p38 (C-20, Santa Cruz Biotechnology).

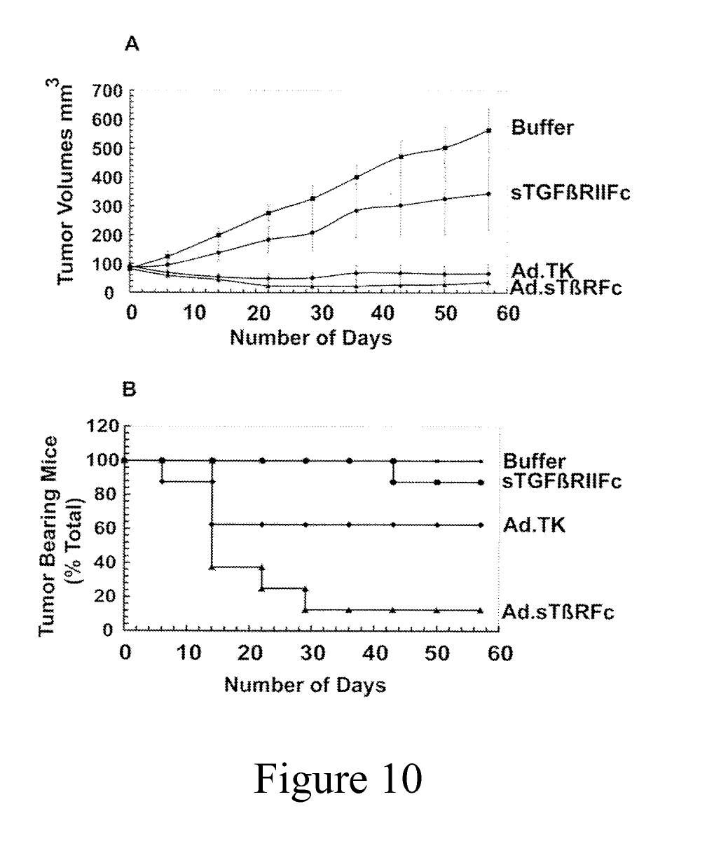

To test whether sTGFβRII could bind to TGF-β, cells were infected with adenoviruses (100 plaque-forming units/cell for 24 hours). sTGFβRII is known to bind with TGF-β1 with much higher affinity compared with TGF-β2 (33). The culture media from uninfected or infected cells were incubated with pure recombinant TGF-β1 and mixed with wheat germ agglutinin-Sepharose beads, which bind to glycosylated proteins, including soluble TGF-β receptor. Beads were washed and subjected to Western blot analysis and probed with anti-TGF-β1 antibody. TGF-β1 was clearly detectable in the precipitate from the medium of soluble TGF-β receptor expressing cells but not from the uninfected or AdNull-infected cells (FIG. 10A). These results indicate that the secreted soluble TGF-β receptor can bind with TGF-β.

Effects on p38 MAPK Activation by Cultured Media from Virus-Infected Cells

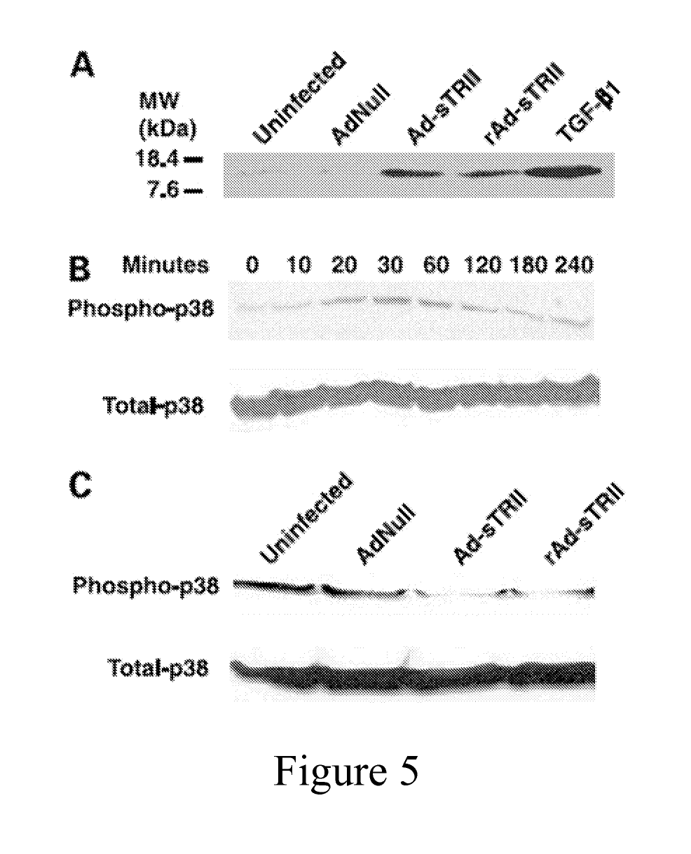

The binding of soluble TGF-β receptor to TGF-β was found to abolish TGF-β signaling in breast cancer cells. Although several biochemical pathways are involved in TGF-β signaling, the p38 MAPK pathway was investigated because it is known to be involved in TGF-β signaling in MDA-MB-231 cells (34). The activation of p38 MAPK by TGF-β in was examined using Western blot assays. Antibodies specific for nonphosphorylated and phosphorylated p38 MAPK were used. As shown in FIG. 5B, the phosphorylation of p38 MAPK was increased in MDA-MB-231 cells after TGF-β1 addition to the media with maximal activation at 30 to 60 minutes. To test whether soluble TGF-β receptor produced by virus-infected cells can functionally inhibit TGF-β activities, we assessed its effect on p38 MAPK phosphorylation in MDA-MB-231 cells. MDA-MB-231 cells cultured in serum-free medium secrete multiple growth factors and cytokines, including TGF-β (35). Cells were infected with 100 MOI of either Ad-sTRII or rAd-sTRII for 24 hours. Culture media from the virally infected cells were collected and centrifuged at 180,000×g. Under these conditions, adenoviruses are known to sediment at the bottom of the centrifuge tube (36). The overnight culture media from uninfected or virus-infected cells were used to treat new set of MDA-MB-231 cells for 1 hour. The cells treated with the culture media from both Ad-sTRII- and rAd-sTRII-infected cells exhibited decreased phosphorylation of p38 MAPK compared with cells treated with media from AdNull-infected or uninfected cells (FIG. 5C). These results indicate that the binding of TGF-β by sTGFβRII in the culture media prevented the maximal activation of p38 MAPK.

MDA-MB-231 cells were incubated with 100 MOI of different adenoviruses for 3 hours in normal growth medium. Cells were washed and incubated in serum-free DMEM for 20 hours. Culture medium was collected and centrifuged at 180,000×g to remove contaminating adenovirus in the medium. A sample (0.1 mL) of this culture medium was mixed with 0.7 mL DMEM without FBS and transferred to serum-starved MDA-MB-231 cells and incubated at 37° C. for 1 hour. Cells were washed, dissolved in SDS sample buffer, and subjected to Western blot analyses to assess p38 MAPK activation.

### Cytotoxicity Assay

Cells were plated in triplicate in 96-well dishes (500 per well) and incubated for 24 hours at 37° C. Cells were exposed to varying concentrations of Ad-sTRII and rAd-sTRII and incubated for an additional 7 days at 37° C. A colorimetric assay was done as described previously (28). Briefly, cells were fixed in 10% trichloroacetic acid for 1 hour, washed five times with water, and allowed to air dry. Cells were then stained for 10 minutes with 0.4% sulforhodamine B (Sigma), dissolved in 1% acetic acid, and rinsed five times with 1% acetic acid. Absorbance (A564ₙₘ) was measured using Spectramax 250 (Molecular Devices, Sunnyvale, Calif.), which was used as a measure of cell number. The IC₅₀ (viral dose that caused 50% cytotoxicity) was calculated assuming the survival rate of uninfected cells to be 100%. The ratio of IC₅₀ was calculated by dividing the IC₅₀ of cells infected with Ad-sTRII by the IC₅₀ of cells infected with rAd-sTRII for each cell line.

### Example 7

### Ad.sTβRFc is Replication Competent in Human Breast Tumor Cells

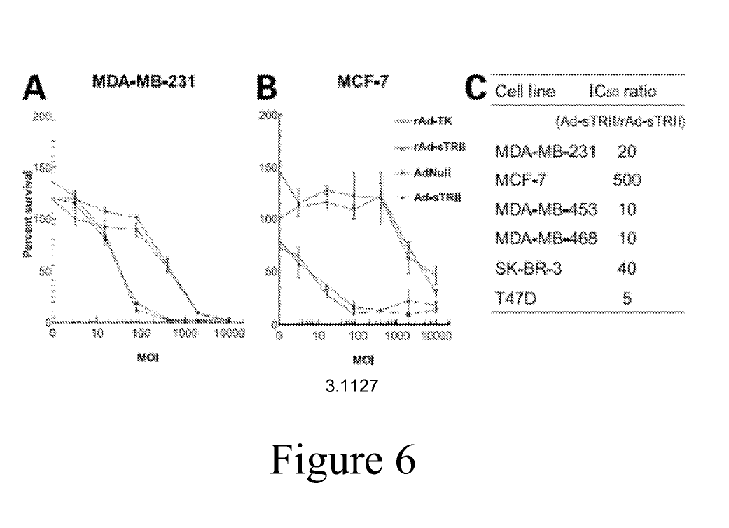

Replication potentials of the oncolytic adenoviruses encoding sTGFβRII or derivatives thereof (e.g., sTGFβRIIFc) were assessed by measuring viral titers in Ad.sTβRFc-infected tumor cells. To assay for viral replication, MDA-MB-231 cells were plated in six-well plates at about 70% confluence and then infected with Ad-sTRII, rAd-sTRII, rAd-TK, or dl309 for 3 hours at an MOI of 50, washed once with DMEM, and incubated in 1 mL DMEM for additional 1 hour at 37° C. At the end of the incubation, cells were washed and divided into two groups. In one group, cells were collected in 0.5 mL growth medium and frozen at −70° C. In the second group, cells were maintained in growth medium for an additional 48 hours. Media and cells in both groups were collected, and cells were subjected to three cycles of freezing and thawing to release the viruses. Total viruses from media and cells were serially diluted and separately added to monolayers of 293 cells. After 3 hours of incubation at 37° C., the infected 293 cells were overlaid with 3 mL 1.25% SeaPlaque agarose (Cambrex, East Rutherford, N.J.) in growth medium. Plaques were counted following 7 to 10 days of incubation using conventional, published methods (29). For cancer therapy purposes, it is important that rAd-sTRII-mediated production of soluble TGF-βRII does not compromise viral replication in the target cells. The effect of adenoviral infections on viral replication was therefore investigated in two different assays: an indirect cytotoxicity assay and a direct method to evaluate the viral titers. To assess viral-mediated cytotoxicity, several breast tumor cell lines were exposed to varying doses of adenoviruses shown in FIGS. 6A and B. The cytotoxicity assays were performed as described herein. In MDA-MB-231 cells, rAd-sTRII caused a dose-dependent increase in cytotoxicity and markedly inhibited cell growth even at viral dosage levels <100 MOI. Under similar conditions, much higher doses of Ad-sTRII were required to induce comparable cytotoxicity. Similarly, rAd-sTRII was relatively more cytotoxic than Ad-sTRII in MCF-7 breast cancer cells. To investigate the contribution of sTGFβRII in cell killing, the effect of rAd-sTRII on cell killing was compared to the cell killing of a control replicating adenovirus, rAd-TK. Both viruses exerted cytotoxic effects on cells in a similar dose-dependent manner. In addition, the basal level toxicity of first-generation E1-deleted adenovirus expressing sTGFβRII was also compared to the basal toxicity of a control E1-deleted adenovirus devoid of any transgene. In this comparison, nearly equal cytotoxic effects on tumor cells was also observed. Thus, sTGFβRII overexpression did not enhance the cytotoxicity of oncolytic or replication-deficient adenoviruses. FIG. 6C shows the ratio of IC₅₀ caused by Ad-sTRII and rAd-sTRII in different breast tumor cell lines. These marked differences in cytotoxicity (5- to 500-fold) inflicted by rAd-sTRII were presumably the result of virus replication in these cancer cells.

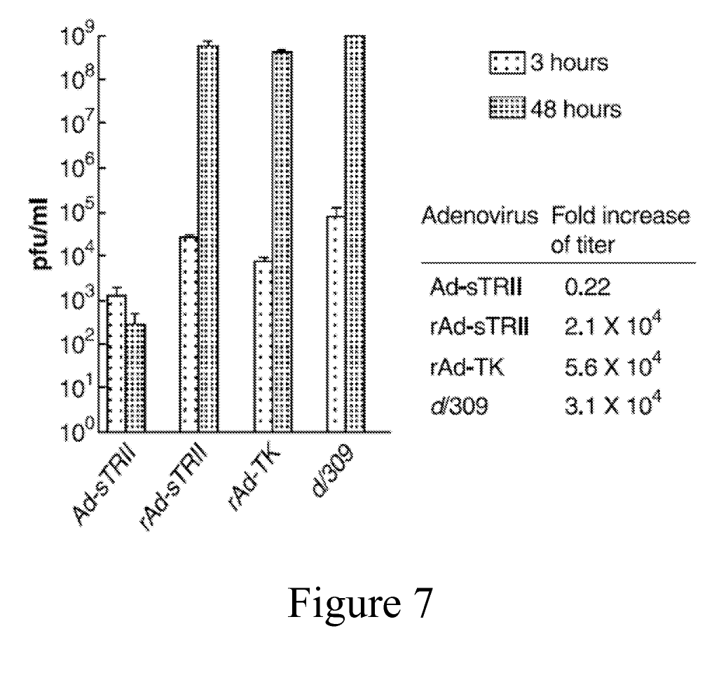

To assess the replication ability of rAd-sTRII in a direct assay, the viral production of rAd-sTRII was compared to that of Ad-sTRII and two control adenoviruses, rAd-TK and dl309, in MDA-MB-231 cells. Total viral particles in the culture medium and in cell fractions were determined by performing plaque assay on 293 cells. After 48 hours of virus infection, viral yields increased significantly (about 4 log differences compared with that of a 3-hour incubation) for rAd-sTRII, rAd-TK, and dl309 adenoviruses (FIG. 7). The titer of rAd-sTRII was only slightly lower than that of rAd-TK and was comparable to that of dl309. In contrast, the titer for replication-deficient Ad-sTRII did not increase but rather slightly decreased after a 48-hour incubation, indicating the inability of Ad-sTRII to replicate in MDA-MB-231 cells. These results indicate that the expression of sTGFβRII does not discernibly inhibit the replication of rAd-sTRII in MDA-MB-231 cells.

Human breast tumor cells were exposed to either Ad.sTβRFc or two control adenoviruses known in the art—a replication-deficient Ad(E1-).Null virus and a fully replication-competent wild type adenovirus dl309—for 48 hours. Cell lysates were prepared and various aliquots were used to evaluate the viral titers by plaque forming assays in HEK 293 cells. More particularly, to assay for viral replication, cells were plated in six-well plates (2×10⁵ cells/well), and infected with Ad(E1-).Null, Ad.sTβRFc, or dl309 (100 pfu/cell) for 3 hours, washed once with DMEM, and incubated in 1 ml DMEM at 37° C. One hour later, cells were washed and were then incubated in growth media for 48 hours. Media and cells were collected and frozen-thawed three times to release the viruses. The lysates were then serially diluted and added to monolayers of HEK 293 cells. After 3 hours of incubation at 37° C., the infected HEK 293 cells were overlaid with 3 ml of 1.25% SeaPlaque agarose in growth media. Plaques were counted following 7 days of incubation.

In MDA-MB-231 cells, the Ad(E1-).Null titer was 1.1×10⁶ pfu (burst size of 1.0). The wild-type dl309 virus titer reached to 2.7×10⁹ pfu/ml, a 2,454-fold increase in burst size (FIG. 9D). The Ad.sTβRFc titer reached 1.12×10⁹ pfu/ml, which represents a 1,018-fold increase in the burst size. Thus, Ad.sTβRFc viral titer was comparable to that of dl309. In MCF-7 breast tumor cells, the Ad.sTβRFc viral titer was 9.7×10⁸ pfu/ml, and 76-NE (Dimri et al., 2002) produced 3.98×10⁸ pfu/ml of Ad.sTβRFc titer which was comparable to the dl309 titers in these cells. These results indicate that Ad.sTβRFc is replication competent in human breast tumor cells.

### Example 8

### Direct Injection into MDA-MB-231 Breast Tumors in Nude Mice Inhibits Tumor Growth and Induces Tumor Regression

The effects of Ad.sTβRFc in a MDA-MB-231 xenograft model were also examined. MDA-MB-231 tumors were established subcutaneously in nude mice. Once the visible tumors (about 80 mm³) appeared, Ad.sTβRFc, Ad.TK, purified sTGFβRIIFc peptide, or buffer alone were injected directly into the tumors. Three injections of viruses (2×10⁸ pfu per injection), or 2.5 μg of peptide per injection, were administered on alternate days (days 0, 2, and 4). Tumor sizes were monitored weekly using a digital caliper. MDA-MB-231 cells were injected subcutaneously (5×10⁶ cells per mouse) as described earlier (Li et al., 1997; Rakkar et al., 1998). Once palpable tumors were established, oncolytic viruses (Ad.sTβRFc, Ad.TK) were administered intratumorally (2×10⁸ pfu in 0.1 ml buffer per injection, three injections total, one each on days 0, 2 and 4). Another group of animals received sTGFβRIIFc (2.5 μg per injection, three injections total, one each on days 0, 2 and 4). Control animals received buffer alone. Tumor volumes were measured using a digital caliper on the various days shown. Animals were monitored weekly for the presence or absence of tumors. The animal experiments were conducted using protocols approved by Evanston Northwestern Healthcare IUCUC committee.

As shown in FIG. 10A, in animals that received buffer alone, tumors grew rapidly from an average tumor volume of 83 mm³ to 563 mm³ by day 57. In the Ad.sTβRFc group, there was a steady decline in tumor growth. On day 57, the average tumor size was 36 mm³, a 93% reduction in average tumor volume compared to the buffer control group (p<0.0001). More importantly, 7 out of 8 animals that received Ad.sTβRFc had complete tumor regressions by day 28 (FIG. 10B). Ad.TK virus also showed significant inhibition of tumor growth (p<0.0001), but Ad.TK treatment resulted in only 3 out of 8 tumor regressions. sTGFβRIIFc peptide alone had a slight effect on tumor growth (p=0.16 versus the buffer group), causing 1 out of 8 tumor regressions. These studies indicate that the anti-tumor effects of Ad.sTβRFc are due, in part, to viral replication, but vector-mediated protein production in combination with viral replication will be useful in treating primary tumors and in treating or preventing metastases. Since the initial proposal of using conditionally replicating adenovirus dl1520 (Onyx 15), which has a mutation in E1b 55 kd protein and replicates in cancer cells that lack wild type p53 gene (Bischoff et al., 1996), several adenoviruses that replicate in cancer cells harboring other genetic defects, such as Rb mutations or deregulated E2F activity have also been used (Heise et al., 2000; DeWeese et al., 2001; Biederer et al., 2002; Johnson et al., 2002; Reid et al., 2002a; Reid et al., 2002b; Post et al., 2003; Yan et al., 2003; Wang et al., 2006). Given that dl01/07-based oncolytic viruses have the capacity to replicate in cancer cells regardless of their genetic background (Howe et al., 1990; Howe et al., 2000), the Ad.sTβRFc will be useful to treat breast cancers of varied genetic constitutions.

### Example 9

### Effect of Intravenous Injection of Ad.TβRIIFc in Breast Tumors Pre-Established in Nude Mice

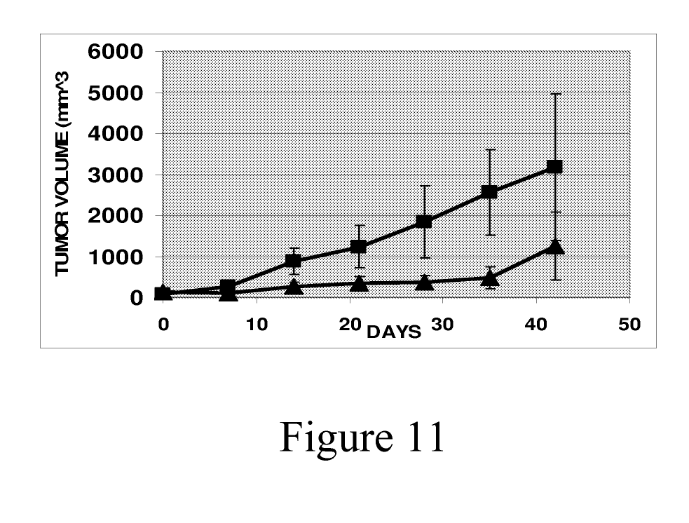

In addition to direct injection, the effects of intravenous administration of Ad.sTβRFc in a MDA-MB-231 xenograft model were examined. Initially, MDA-MB-231 breast tumor xenografts were established in nude mice. Four-week-old nude mice (Nu/Nu) were injected with 10 million cells subcutaneously. After 8-days, when palpable tumors were established, Ad.TβRIIFc (2×10⁸ pfus in 0.1 ml of buffer), or buffer used as a control, was injected intravenously into the tail veins of mice harboring breast tumor xenografts. The intravenous administration of Ad.TβRIIFc (2×10⁸ pfus in 0.1 ml of buffer) or buffer control was repeated three days later. Tumor sizes were monitored once a week. The results shown in FIG. 11 reveal that, in the Ad.TβRIIFc treated group, 30% of the animals were tumor-free on day 42, while all the animals in the control group had large tumors. These results establish that the oncolytic Ad.sTβRFc virus is effective against MDA-MB-231 tumors when administered indirectly to the tumor, i.e., by intravenous administration. It is expected that adenoviruses expressing, and preferably selectively expressing, a protein that specifically binds to TGF-β will be useful in the treatment of oncolytic disease. Exemplary adenoviruses according to the disclosure include adenoviruses expressing a soluble form of the TGF-β receptor-II, alone or fused to a stabilizing peptide such as Fc (sTGFβRIIFc). In preferred embodiments, an adenoviral dl01/07 mutant is used because it can replicate in all cancer cells, regardless of their genetic defects, and because it can accommodate an operative coding region for a protein or peptide that specifically binds to TGF-β, such as the proteins described and/or defined herein.

### REFERENCES

AKHURST, R. J., and DERYNCK, R. (2001). TGF-beta signaling in cancer—a double-edged sword. Trends Cell Biol 11, S44-51.
BIEDERER, C., RIES, S., BRANDTS, C. H., and MCCORMICK, F. (2002). Replication-selective viruses for cancer therapy. J Mol Med 80, 163-175.
BISCHOFF, J. R., KIRN, D E., WILLIAMS, A., HEISE, C., HORN, S., MUNA, M., NG, L., NYE, J. A., SAMPSON-JOHANNES, A., FATTAEY, A., and MCCORMICK, F. (1996). An adenovirus mutant that replicates selectively in p53-deficient human tumor cells. Science 274, 373-376.
CRAIG, C., WERSTO, R., KIM, M., OHM, E., LI, Z., KATAYOSE, D., LEE, S. J., TREPEL, J., COWAN, K., and SETH, P. (1997). A recombinant adenovirus expressing p27Kip1 induces cell cycle arrest and loss of cyclin-Cdk activity in human breast cancer cells. Oncogene 14, 2283-2289.
DERYNCK, R., AKHURST, R. J., and BALMAIN, A. (2001). TGF-beta signaling in tumor suppression and cancer progression. Nat Genet. 29, 117-129.
DEWEESE, T. L., VAN DER POEL, H., LI, S., MIKHAK, B., DREW, R., GOEMANN, M., HAMPER, U., DEJONG, R., DETORIE, N., RODRIGUEZ, R., HAULK, T., DEMARZO, A. M., PIANTADOSI, S., YU, D. C., CHEN, Y., HENDERSON, D. R., CARDUCCI, M. A., NELSON, W. G., and SIMONS, J. W. (2001). A phase I trial of CV706, a replication-competent, PSA selective oncolytic adenovirus, for the treatment of locally recurrent prostate cancer following radiation therapy. Cancer Res 61, 7464-7472.
DIMRI, G. P., MARTINEZ, J. L., JACOBS, J. J., KEBLUSEK, P., ITAHANA, K., VAN LOHUIZEN, M., CAMPISI, J., WAZER, D. E., and BAND, V. (2002). The Bmi-1 oncogene induces telomerase activity and immortalizes human mammary epithelial cells. Cancer Res 62, 4736-4745.
GUISE, T. A., and CHIRGWIN, J. M. (2003). Transforming growth factor-beta in osteolytic breast cancer bone metastases. Clin Orthop, S32-38.
HEISE, C., HERMISTON, T., JOHNSON, L., BROOKS, G., SAMPSON-JOHANNES, A., WILLIAMS, A., HAWKINS, L., and KIRN, D. (2000). An adenovirus Et A mutant that demonstrates potent and selective systemic anti-tumoral efficacy. Nat Med 6, 1134-1139.
HIRAGA, T., WILLIAMS, P. J., MUNDY, G. R., and YONEDA, T. (2001). The bisphosphonate ibandronate promotes apoptosis in MDA-MB-231 human breast cancer cells in bone metastases. Cancer Res 61, 4418-4424.
HOWE, J. A., DEMERS, G. W., JOHNSON, D. E., NEUGEBAUER, S. E., PERRY, S. T., VAILLANCOURT, M. T., and FAHA, B. (2000). Evaluation of E1-mutant adenoviruses as conditionally replicating agents for cancer therapy. Mol Ther 2, 485-495.
HOWE, J. A., MYMRYK, I S., EGAN, C., BRANTON, P. E., and BAYLEY, S. T. (1990). Retinoblastoma growth suppressor and a 300-kDa protein appear to regulate cellular DNA synthesis. Proc Natl Acad Sci USA 87, 5883-5887.
INGE, T. H., HOOVER, S. K., SUSSKIND, B. M., BARRETT, S. K., and BEAR, H. D. (1992). Inhibition of tumor-specific cytotoxic T-lymphocyte responses by transforming growth factor beta 1. Cancer Res 52, 1386-1392.
IWASAKI, T., MUKAI, M., TSUJIMURA, T., TATSUTA, M., NAKAMURA, H., TERADA, N., and AKEDO, H. (2002). Ipriflavone inhibits osteolytic bone metastasis of human breast cancer cells in a nude mouse model. Int J Cancer 100, 381-387.
IYER, I., WANG, Z.-G., AKHTARI, M., ZHAO, W., and SETH, P. (2005). Targeting TGF beta signaling for cancer therapy. Cancer Biol Ther 4, e33-e38.
JOHNSON, L., SHEN, A., BOYLE, L., KUNICH, J., PANDEY, K., LEMMON, M., HERMISTON, T., GIEDLIN, M., MCCORMICK, F., and FATTAEY, A. (2002). Selectively replicating adenoviruses targeting deregulated E2F activity are potent, systemic antitumor agents. Cancer Cell 1, 325-337.
KANAMOTO, T., HELLMAN, U., HELDIN, C H., and SOUCHELNYTSKYI, S. (2002). Functional proteomics of transforming growth factor-beta1-stimulated Mv1Lu epithelial cells: RadS1 as a target of TGFbeta1-dependent regulation of DNA repair. Embo J 21, 1219-1230.
KATAYOSE, D., GUDAS, J., NGUYEN, H., SRIVASTAVA, S., COWAN, K. H., and SETH, P. (1995). Cytotoxic effects of adenovirus-mediated wild-type p53 protein expression in normal and tumor mammary epithelial cells. Clin Cancer Res 1, 889-897.
LI, Z., SHANMUGAM, N., KATAYOSE, D., HUBER, B., SRIVASTAVA, S., COWAN, K., and SETH, P. (1997). Enzyme/prodrug gene therapy approach for breast cancer using a recombinant adenovirus expressing Escherichia coli cytosine deaminase. Cancer Gene Ther 4, 113-117.
POST, D. E., KHURI, F. R., SIMONS, J. W., and VAN MEIR, E. G. (2003). Replicative oncolytic adenoviruses in multimodal cancer regimens. Hum Gene Ther 14, 933-946.
RAKKAR, A. N., LI, Z., KATAYOSE, Y., KIM, M., COWAN, K. H., and SETH, P. (1998). Adenoviral expression of the cyclin-dependent kinase inhibitor p27Kip1: a strategy for breast cancer gene therapy. J Natl Cancer Inst 90, 1836-1838.
REID, T., GALANIS, E., ABBRUZZESE, J., SZE, D., WEIN, L. M., ANDREWS, J., RANDLEV, B., HEISE, C., UPRICHARD, M., HATFIELD, M., ROME, L., RUBIN, J., and KIRN, D. (2002a). Hepatic arterial infusion of a replication-selective oncolytic adenovirus (d11520): phase II viral, immunologic, and clinical endpoints. Cancer Res 62, 6070-6079.
REID, T., WARREN, R., and KIRN, D. (2002b). Intravascular adenoviral agents in cancer patients: lessons from clinical trials. Cancer Gene Ther 9, 979-986.
ROBERTS, A. B., and WAKEFIELD, L. M. (2003). The two faces of transforming growth factor beta in carcinogenesis. Proc Natl Acad Sci USA 100, 8621-8623.
SETH, P., ed. (1999). Adenoviruses : Basic Biology to Gene Therapy. (R G Landes Company, Austin, Tex.).
SETH, P., BRINKMANN, U., SCHWARTZ, G. N., KATAYOSE, D., GRESS, R., PASTAN, I., and COWAN, K. (1996). Adenovirus-mediated gene transfer to human breast tumor cells: an approach for cancer gene therapy and bone marrow purging. Cancer Res 56, 1346-1351.
TANG, B., VU, M., BOOKER, T., SANTNER, S. J., MILLER, F. R., ANVER, M. R., and WAKEFIELD, L. M. (2003). TGF-beta switches from tumor suppressor to prometastatic factor in a model of breast cancer progression. J Clin Invest 112, 1116-1124.
TEICHER, B. A. (2001). Malignant cells, directors of the malignant process: role of transforming growth factor-beta. Cancer Metastasis Rev 20, 133-143.
THOMAS, D. A., and MASSAGUE, J. (2005). TGF-beta directly targets cytotoxic T cell functions during tumor evasion of immune surveillance. Cancer Cell 8, 369-380.
WAKEFIELD, L. M., and ROBERTS, A. B. (2002). TGF-beta signaling: positive and negative effects on tumorigenesis. Curr Opin Genet Dev 12, 22-29.
WANG, Z. G., ZHAO, W., RAMACHANDRA, M., and SETH, P. (2006). An oncolytic adenovirus expressing soluble transforming growth factor-beta type II receptor for targeting breast cancer: in vitro evaluation. Mol Cancer Ther 5, 367-373.
WRANA, J. L., ATTISANO, L., CARCAMO, J., ZENTELLA, A., DOODY, J., LAIHO, M., WANG, X. F., and MASSAGUE, J. (1992). TGF beta signals through a heteromeric protein kinase receptor complex. Cell 71, 1003-1014.
YAN, W., KITZES, G., DORMISHIAN, F., HAWKINS, L., SAMPSON-JOHANNES, A., WATANABE, J., HOLT, J., LEE, V., DUBENSKY, T., FATTAEY, A., HERMISTON, T., BALMAIN, A., and SHEN, Y. (2003). Developing novel oncolytic adenoviruses through bioselection. J Virol 77, 2640-2650.
YANG, Y. A., DUKHANINA, O., TANG, B., MAMURA, M., LETTERIO, J. J., MACGREGOR, J., PATEL, S. C., KHOZIN, S., LIU, Z. Y., GREEN, J., ANVER, M. R., MERLINO, G., and WAKEFIELD, L. M. (2002). Lifetime exposure to a soluble TGF-beta antagonist protects mice against metastasis without adverse side effects. J Clin Invest 109, 1607-1615.
YIN, J. J., SELANDER, K., CHIRGWIN, J. M., DALLAS, M., GRUBBS, B. G., WIESER, R., MASSAGUE, J., MUNDY, G. R., and GUISE, T. A. (1999). TGF-beta signaling blockade inhibits PTHrP secretion by breast cancer cells and bone metastases development. J Clin Invest 103, 197-206.
ZHAO, W., KOBAYASHI, M., DING, W., YUAN, L., SETH, P., CORNAIN, S., WANG, J., OKADA, F., and HOSOKAWA, M. (2002). Suppression of in vivo tumorigenicity of rat hepatoma cell line KDH-8 cells by soluble TGF-beta receptor type II. Cancer Immunol Immunother 51, 381-388.
1. Seth P, editor. Adenoviruses: basic biology to gene therapy. Austin (TX): R G Landes Company; 1999. p. 1-314.
2. Seth P. Adenoviral vectors. Adv Exp Med Biol 2000; 465:13-22.
3. Hitt M M, Graham F L. Adenovirus vectors for human gene therapy. Adv Virus Res 2000; 55:479-505.
4. Imperiale M J, Kochanek S. Adenovirus vectors: biology, design, and production. Curr Top Microbiol Immunol 2004; 273:335-57.
5. Bischoff J R, Kim D H, Williams A, et al. An adenovirus mutant that replicates selectively in p53-deficient human tumor cells. Science 1996; 274:373-6.
6. Biederer C, Ries S, Brandts C H, McCormick F. Replication-selective viruses for cancer therapy. J Mol Med 2002; 80:163-75.
7. Post D E, Khuri F R, Simons J W, Van Meir E G. Replicative oncolytic adenoviruses in multimodal cancer regimens. Hum Gene Ther 2003; 14: 933-46.
8. Howe J A, Mymryk J S, Egan C, Branton P E, Bayley S T. Retinoblastoma growth suppressor and a 300-kDa protein appear to regulate cellular DNA synthesis. Proc Natl Acad Sci USA 1990; 87:5883-7.
9. Muraoka-Cook R S, Dumont N, Arteaga C L. Dual role of transforming growth factor beta in mammary tumorigenesis and metastatic progression. Clin Cancer Res 2005; 11:937-43s .
10. Roberts A S, Wakefield L M. The two faces of transforming growth factor beta in carcinogenesis. Proc Natl Acad Sci USA 2003; 100:8621-3.
11. Akhurst R J, Derynck R. TGF-beta signaling in cancer: a double-edged sword. Trends Cell Biol 2001; 11:S44-51.
12. Inge T H, Hoover S K, Susskind B M, Barrett S K, Bear H D. Inhibition of tumor-specific cytotoxic T-lymphocyte responses by transforming growth factor beta 1. Cancer Res 1992; 52:1386-92.
13. Wojtowicz-Praga S. Reversal of tumor-induced immunosuppression by TGF-beta inhibitors. Invest New Drugs 2003; 21:21-32.
14. Iyer I, Wang Z-G, Akhtari M, Zhao W, Seth P. Targeting TGF beta signaling for cancer therapy. Cancer Biol Ther 2005; 4:e33-8.
15. Guise T A, Chirgwin J M. Transforming growth factor-beta in osteolytic breast cancer bone metastases. Clin Orthop Relat Res 2003; 532-8.
16. Yin J J, Selander K, Chirgwin J M, et al. TGF-beta signaling blockade inhibits PTHrP secretion by breast cancer cells and bone metastases development. J Clin Invest 1999; 103:197-206.
17. Ishida A, Fujita N, Kitazawa R, Tsuruo T. Transforming growth factor-beta induces expression of receptor activator of NF-kappa B ligand in vascular endothelial cells derived from bone. J Biol Chem 2002; 277: 26217-24.
18. Teicher B A. Malignant cells, directors of the malignant process: role of transforming growth factor-beta. Cancer Metastasis Rev 2001; 20: 133-43.
19. Muraoka R S, Dumont N, Ritter C A, et al. Blockade of TGF-beta inhibits mammary tumor cell viability, migration, and metastases. J Clin Invest 2002; 109:1551-9.
20. Massague J, Wotton D. Transcriptional control by the TGF-beta/Smad signaling system. EMBO J. 2000; 19:1745-54.
21. Siegel P M, Massague J. Cytostatic and apoptotic actions of TGF-beta in homeostasis and cancer. Nat Rev Cancer 2003; 3:807-21.
22. Yingling J M, Blanchard K L, Sawyer J S. Development of TGF-beta signalling inhibitors for cancer therapy. Nat Rev Drug Discov 2004; 3: 1011-22.
23. Dumont N, Arteaga C L. Targeting the TGF beta signaling network in human neoplasia. Cancer Cell 2003; 3:531-6.
24. Zhao W, Kobayashi M, Ding W, at al. Suppression of in vivo tumorigenicity of rat hepatoma cell line KDH-8 cells by soluble TGF-beta receptor type II. Cancer Immunol Immunother 2002; 51:381-8.
25. He T C, Zhou S, da Costa L T, Yu J, Kinzler K W, Vogelstein B. A simplified system for generating recombinant adenoviruses. Proc Natl Acad Sci USA 1998; 95:2509-14.
26. Li Z, Shanmugam N, Katayose D, et al. Enzyme/prodrug gene therapy approach for breast cancer using a recombinant adenovirus expressing Escherichia coli cytosine deaminase. Cancer Gene Ther 1997; 4:113-7.
27. Bett A J, Krougliak V, Graham F L. DNA sequence of the deletion/insertion in early region 3 of Ad5 dl309. Virus Res 1995; 39:75-82.
28. Katayose D, Gudas J, Nguyen H, Srivastava S, Cowan K H, Seth P. Cytotoxic effects of adenovirus-mediated wild-type p53 protein expression in normal and tumor mammary epithelial cells. Clin Cancer Res 1995; 1:889-97.
29. Seth P, Higginbotham J. Advantages and disadvantages of multiple different methods of adenoviral vector construction. In: Habib N, editor. Methods in molecular medicine. Vol. 45. Totowa (NJ): Humana Press, Inc; 2000. p. 189-98.
30. Craig C, Wersto R, Kim M, et al. A recombinant adenovirus expressing p27Kip1 induces cell cycle arrest and loss of cyclin-Cdk activity in human breast cancer cells. Oncogene 1997; 14:2283-9.
31. Lin H Y, Moustakas A, Knaus P, Wells R G, Henis Y l, Lodish H F. The soluble exoplasmic domain of the type II transforming growth factor (TGF)-beta receptor. A heterogeneously glycosylated protein with high affinity and selectivity for TGF-beta ligands. Biol Chem 1995; 270: 2747-54.
32. Maley F, Trimble R B, Tarentino A L, Plummer T H, Jr. Characterization of glycoproteins and their associated oligosaccharides through the use of endoglycosidases. Anal Biochem 1989; 180:195-204.
33. Tsang M L, Zhou L, Zheng B L, et al. Characterization of recombinant soluble human transforming growth factor-beta receptor type II (rhTGF-beta sR11). Cytokine 1995; 7:389-97.
34. Kakonen S M, Selander K S, Chirgwin J M, et al. Transforming growth factor-beta stimulates parathyroid hormone-related protein and osteolytic metastases via Smad and mitogen-activated protein kinase signaling pathways. J Biol Chem 2002; 277:24571-8.
35. Pederson L, Winding B, Foged N T, Spelsberg T C, Oursler M J. Identification of breast cancer cell line-derived paracrine factors that stimulate osteoclast activity. Cancer Res 1999; 59:5849-55.
36. Seth P. Mechanism of adenovirus-mediated endosome lysis: role of the intact adenovirus capsid structure. Biochem Biophys Res Commun 1994; 205:1318-24.
37. Roth J A, Grammer S F. Tumor suppressor gene therapy. Methods Mol Biol 2003; 223:577-98.
38. Ivanovic V, Todorovic-Rakovic N, Demajo M, at al. Elevated plasma levels of transforming growth factor-beta 1 (TGF-beta 1) in patients with advanced breast cancer: association with disease progression. Eur J Cancer 2003; 39:454-61.
39. Gold L I. The role for transforming growth factor-beta (TGF-beta) in human cancer. Crit. Rev Oncog 1999; 10:303-60.
40. Howe J A, Demers G W, Johnson D E, at al. Evaluation of E1-mutant adenoviruses as conditionally replicating agents for cancer therapy. Mol Ther 2000; 2:485-95.

Each of the references cited herein is incorporated by reference in its entirety.

Variations on the subject matter disclosed herein will be apparent to those of skill in the art upon review of the present disclosure, and such variant subject matter is within the scope of the invention.

What is claimed is:

1. A method for treating cancer comprising delivering a therapeutically effective amount of an adenovirus expressing an E1A protein deficient in facilitating host cell transition to the S phase of growth and further comprising a coding region for a protein that specifically binds to TGF-β.

2. The method according to claim 1 wherein the adenovirus comprises a mutated E1A coding region.

3. The method according to claim 1 wherein the adenovirus is Ad dl01/07 comprising a coding region for a protein that specifically binds to TGF-β.

4. The method according to claim 1 wherein the coding region encodes a TGF-β receptor.

5. The method according to claim 4 wherein the TGF-β receptor is TGF-β receptor II.

6. The method according to claim 5 wherein the TGF-β receptor II is a soluble TGF-β receptor II.

7. The method according to claim 1 wherein the protein that specifically binds to TGF-β is a fusion protein.

8. The method according to claim 7 wherein the fusion protein is a fusion between a TGF-β receptor and an F_{C} fragment of an immunoglobulin.

9. The method according to claim 8 wherein the F_{C} fragment is an IgG F_{C} fragment.

10. The method according to claim 8 wherein the IgG F_{C} fragment is an IgG1 F_{C} fragment.

11. A method for treating cancer comprising delivering a therapeutically effective amount of an adenovirus comprising an essential adenoviral gene under the expression control of a tumor-specific promoter and further comprising a coding region for a protein that specifically binds to TGF-β.

12. The method according to claim 11 wherein the tumor-specific promoter is selected from the group consisting of an hTERT promoter, a modified hTERT promoter and a promoter for a small mucin-like protein.

13. The method according to claim 12 wherein the protein that specifically binds to TGF-β is selected from the group consisting of TGF-β receptor I, TGF-β receptor II and soluble TGF-β receptor II.

14. A method for treating cancer comprising delivering a therapeutically effective amount of an adenovirus comprising a coding region for a fusion protein comprising an adenoviral fiber protein and a binding pair member that specifically interacts with a binding partner associated with a cancer cell, and further comprising a coding region for a protein that specifically binds to TGF-β.

15. The method according to claim 14 wherein the binding pair member is selected from the group consisting of Lyp-1, RGD-4C, NGR and F-3.

16. The method according to claim 15 wherein the protein that specifically binds to TGF-β is selected from the group consisting of TGF-β receptor I, TGF-β receptor II and soluble TGF-β receptor II.

17. Use of an adenovirus expressing an E1A protein deficient in facilitating host cell transition to the S phase of growth and further comprising a coding region for a protein that specifically binds to TGF-β in the preparation of a medicament for the treatment of cancer.

18. An adenovirus expressing an E1A protein deficient in facilitating host cell transition to the S phase of growth and further comprising a coding region for a protein that specifically binds to TGF-β.

19. The adenovirus according to claim 18 in combination with a pharmaceutically acceptable diluent, carrier or excipient, the adenovirus and pharmaceutically acceptable diluent, carrier or excipient constituting a pharmaceutical composition.

20. The pharmaceutical composition according to claim 19 in combination with a protocol for administration, the pharmaceutical composition and protocol constituting a kit.

21. The method according to claim 1 wherein the adenovirus is administered by intravenous injection.

22. The method according to claim 14 wherein the adenovirus is administered by intravenous injection.
